(19) United States
(12) Patent Application Publication
(10) Pub. No.: US 2017/0182310 A1
(43) Pub. Date: Jun. 29, 2017

(54) PROCESS FOR ELECTRICALLY CONTACTING A COATED LEAD WITH A PARTICLE

(71) Applicant: Heraeus Deutschland GmbH & Co. KG, Hanau (DE)

(72) Inventors: Jens TROETZSCHEL, Ronneburg (DE); Stefan SCHIBLI, Frankfurt (DE)

(73) Assignee: Heraeus Deutschland GmbH & Co. KG, Hanau (DE)

(21) Appl. No.: 15/388,913

(22) Filed: Dec. 22, 2016

(30) Foreign Application Priority Data
Dec. 23, 2015 (EP) 15202411.3

(51) Int. Cl. A61N 1/05; A61B 5/00

(52) U.S. Cl. CPC A61N 1/05; A61B 5/6846; A61N 1/0551; A61B 2562/125

(57) ABSTRACT

One aspect relates generally to a method for electrically contacting a coated lead. One aspect relates to a method for electrically contacting a coated lead including providing a coated lead including an electrically conductive core and an electrically insulating coating. A via hole is provided in the electrically insulating coating in order to expose a section of the electrically conductive core. A first electrically conductive material is introduced into the via hole such that it contacts at least a part of the exposed section of the electrically conductive core. A further electrically conductive material is applied to the coated lead such that it contacts at least a part of the first conductive material.

### CROSS-REFERENCE TO RELATED APPLICATIONS

This Utility patent application claims priority to European Patent Application No. EP 15202411.3, filed on Dec. 23, 2015, which is incorporated herein by reference.

### BACKGROUND

In general, one aspect relates generally to a process for electrically contacting a coated lead. More specifically, one aspect relates to a process, an electrically contacted lead and a medical device including an electrically contacted lead. Making electrical contact with an electrically conductive lead can often represent a significant challenge. In order to take advantage of beneficial properties of an electrical lead, such as good electrical conductivity and good durability, the contact to the lead itself must also display similar beneficial properties.

Such considerations are particularly pertinent for example when electrically contacting leads in medical devices. In devices which are introduced into the body, it is desirable to employ thin leads, which can represent a challenge to electrically contact due their size. Furthermore, a very high value is placed on reliability in medical devices such as Cardiac Pacemakers, Implantable Cardioverter Defibrillation Devices and Cardiac Resynchronisation Devices, especially in terms of resistance to physical fatigue. Invasive surgery is required to implant medical devices into the body or remove or replace parts, and it is highly desirable for the individual components of the device to have a long working life in order to reduce the requirement for surgical intervention. Furthermore, it is desirable for the working life to have a low variance. One component of a medical device which is exposed to a particularly high amount of stress during normal operation is the lead and the electrical connections thereto.

Document U.S. Pat. No. 7,364,479 B1 discloses a method for contacting a lead by crimping. Direct crimping can result in a contact which is lost over time due to physical movement. Furthermore, the direct crimping method is not suitable for multi-core leads since contact would be made to the cores indiscriminately.

Document US 2013/0338745 A1 discloses a method for contacting individual conductive cores of a cable which includes multiple conductive cores. The individually coated conductive cores are fed through the lumens of a ring, and electrical contact is made with the individual cores by piercing. This method suffers at least from the disadvantages associated with moveable parts.

For these and other reasons, a need exists for the present invention.

### SUMMARY

One aspect is generally based on the object of overcoming at least one of the problems encountered in the state of the art in relation to electrical contacts with leads.

One embodiment is providing a process for electrically contacting a lead, which provides for one or more selected from the following group of advantages: an improved electrical contact, an improved durability and a more flexible process.

One embodiment provides a process that is suitable for electrically contacting a thin lead, while providing for one or more selected from the following group of advantages: an improved electrical contact, an improved durability and a more flexible process.

Another embodiment provides a process that is suitable for electrically contacting a lead for use in a medical device, in one embodiment, a thin lead. One embodiment electrically contacts a lead in a bio-compatible device, while providing for one or more selected from the following group of advantages: an improved electrical contact, an improved durability and a more flexible process. One aspect of this embodiment provides a process for connecting a lead which fulfils the requirements laid out in the “Guidance for the Submission of Research and Marketing Applications for Permanent Pacemaker Leads and for Pacemaker Lead Adaptor 510(k) Submissions” issued by the “Center for Devices and Radiological Health” of the US Food and Drug Administration. A further object is provide a method for electrically contacting a multi-core lead, for example, for discriminately contacting a single core thereof. This has long been attempted and there are various approaches provided in the art. Some of these have been discussed above. There remains, however, a need for further improvement of such electrical contacts and methods for making them.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

Embodiments are now further illustrated using figures which are not to be considered as limiting in scope. In brief, the figures illustrate the following:

FIGS. 1a to 1d illustrate schematically the process for electrically contacting a coated lead.

FIG. 2 illustrates schematically a process according to one embodiment for contacting a coated lead.

FIG. 3 illustrates schematically a pacemaker including a lead electrically connected according to one embodiment.

FIG. 4 illustrates schematically a lead with 3 electrically conductive cores, contacted six times according to one embodiment.

FIG. 5 illustrates a scanning electron microscope image of electrically conductive particles according to one embodiment.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

A contribution to achieving at least one of the above described objects is made by the subject matter of the category forming claims. A further contribution is made by the subject matter of the dependent claims, which represent specific embodiments.

A contribution to achieving at least one of the above described objects is made by the following embodiments.
1. Process for electrically contacting a coated lead including the following steps:
   a. Providing a coated lead including an electrically conductive core and an electrically insulating coating;
   b. Providing a via hole in the electrically insulating coating in order to expose a section of the electrically conductive core;
   c. Introducing a first electrically conductive material into the via hole such that it contacts at least a part of the exposed section of the electrically conductive core;
   d. Applying a further electrically conductive material to the coated lead such that it contacts at least a part of the first conductive material.
2. The process according to embodiment 1, wherein the first electrically conductive material includes an electrically conductive particle, in one embodiment a micro-particle. In one aspect of this embodiment, the first electrically conductive material is one or more electrically conductive particles, in one embodiment is one conductive particle. In one aspect of this embodiment, the first electrically conductive material includes 1 to 20 electrically conductive particles, in one embodiment 1 to 5 conductive particles, in one embodiment 1 or 2 conductive particles, or in one embodiment one conductive particle.
3. The process according to embodiment 2, wherein the particle has a diameter in the range from about 5 to about 500 μm, in one embodiment in the range from about 10 to about 200 μm, in one embodiment in the range from about 20 to about 100 μm, or in one embodiment in the range from about 30 to about 70 μm.
4. The process according to embodiment 2 or 3, wherein the particle has a diameter which is greater than the thickness of the electrically insulating coating, in one embodiment a diameter in the range from more than about 1 to about 4 times, in one embodiment in the range from about 1.2 to about 3 times, further in one embodiment from about 1.3 to about 2.7 times, or in one embodiment from about 1.4 to about 2.3 times the thickness of the coating.
5. The process according to any of the embodiments 2 to 4, wherein the particle is spherical.
6. The process according to any of the embodiments 2 to 5, wherein the introduction of the particle includes sprinkling, dipping, dusting, the use of tweezers, or a combination of two or more selected therefrom, wherein dipping is preferred in one embodiment.
7. The process according to any of the preceding embodiments, wherein the first electrically conductive material has a mass in the range from about 0.01 to about 20 μg, in one embodiment in the range from about 0.05 to about 10 μg, in one embodiment in the range from about 0.08 to about 8 μg, or in one embodiment in the range from about 0.1 to about 5 μg.
8. The process according to any of the preceding embodiments, wherein the first electrically conductive material includes a metal.
9. The process according to any of the preceding embodiments, wherein the first electrically conductive material is the same material as the electrically conductive core.
10. The process according to any of the preceding embodiments, wherein the second electrically conductive material is applied by solid deformation.
11. The process according to any of the preceding embodiments, wherein the application of the second electrically conductive material in step d. includes one or more selected from the group consisting of: crimping, notching, bending, twisting, screwing and press fitting, in one embodiment crimping.
12. The process according to any of the preceding embodiments, wherein the second electrically conductive material is a metal.
13. The process according to any of the preceding embodiments, wherein the lead has a diameter less than about 2 mm, in one embodiment less than about 1.5 mm, in one embodiment less than about 1 mm, or in one embodiment less than about 0.7 mm. In some cases, the thickness of the lead is as little as 0.1 mm or more.
14. The process according to any of the preceding embodiments, wherein the electrically conductive core is a metal.
15. The process according to any of the preceding embodiments, wherein the electrically insulating coating is a polymer.
16. The process according to any of the preceding embodiments, wherein the lead includes 2 or more electrically conductive cores, in one embodiment 3 or more, or in one embodiment 4 or more.
17. The process according to embodiment 16, wherein 2 or more of the electrically conductive cores are electrically insulated from each other by an insulated material, in one embodiment each of the conductive cores is insulated from each of the other conductive cores.
18. The process according to any of the preceding embodiments, wherein the via hole exposes a single conductive core.
19. The process according to any of the preceding embodiments, wherein the section of the electrically conductive core exposed by the via hole has a surface area in the range from about 0.001 to about 0.1 mm², in one embodiment in the range from about 0.005 to about 0.08 mm², or in one embodiment in the range from about 0.008 to about 0.04 mm².
20. The process according to any of the preceding embodiments, wherein the provision of the via hole in step b. is performed with a laser.
21. An electrically contacted lead obtainable by a method according to any of the preceding embodiments.
22. An electrically contacted lead including the following:
   a. an electrically conductive core
   b. an electrically insulating coating which coats the electrically conductive core;
   c. a via hole in the electrically insulating coating which exposes a section of the electrically conductive core;
   d. a first electrically conductive material in the via hole which contacts at least a part of the exposed section of the electrically conductive core;
   e. a further electrically conductive material which contacts at least a part of the first conductive material.

The features introduced in relation to the above process embodiments apply mutatis mutandis as aspects of this embodiment.
23. A medical device including a contacted lead according to embodiment 21 or 22.
24. A medical device according to embodiment |23|, selected from the group consisting of: a pacemaker, a neuro-stimulator, a measuring device, and a defibrillator.
25. A use of electrically conductive particles for contacting a coated lead, wherein the electrically conductive particles satisfy one or more of the following criteria:
   a. D₅₀ in the range from about 10 to about 100 μm, in one embodiment in the range from about 15 to about 60 μm, or in one embodiment in the range from about 20 to about 50 μm;
b. D₁₀ in the range from about 5 to about 40 μm, in one embodiment in the range from about 10 to about 35 μm, or in one embodiment in the range from about 15 to about 30 μm;
C. D₉₀ in the range from about 40 to about 70 μm, in one embodiment in the range from about 45 to about 65 μm, or in one embodiment in the range from about 50 to about 60 μm;
d. Difference D₉₀-D₁₀ in the range from about 1 to about 25 μm, in one embodiment in the range from about 3 to about 15 μm, more in or one embodiment in the range from about 5 to about 10 μm;
e. A standard deviation of diameter in the range from about 0.5 to about 10 μm, in one embodiment in the range from about 1 to about 8 μm, or in one embodiment in the range from about 2 to about 5 μm.

Aspects of these embodiments, the following combinations are satisfied: a, b, c, d, a+b, a+c, a+d, b+c, b+d, c+d, a+b+c, a+b+d, a+c+d, b+c+d, a+b+c+d, e, a+e, b+e, c+e, d+e, a+b+e, a+c+e, a+d+e, b+c+e, b+d+e, c+d+e, a+b+c+e, a+b+d+e, a+c+d+e, b+c+d+e or a+b+c+d+e. The features of the first electrically conductive material are preferred for the electrically conductive particles in one embodiment.

Process for Electrically Contacting a Lead

A contribution to achieving at least one of the above mentioned objects is made by a process for electrically contacting a lead. The process according to one embodiment includes the following steps:
a. Providing a coated lead including an electrically conductive core and an electrically insulating coating;
b. Providing a via hole in the electrically insulating coating in order to expose a section of the electrically conductive core;
c. Introducing a first electrically conductive material into the via hole such that it contacts at least a part of the exposed section of the electrically conductive core;
d. Applying a further electrically conductive material to the coated lead such that it contacts at least a part of the first conductive material.

In one embodiment, the lead is electrically contacted once by the process according to one embodiment. In another embodiment, the lead is electrically contacted two or more times by the process according to one embodiment.

The lead of step a. is the lead to be contacted. The conductive core is in one embodiment longitudinal, extending along the lead. The electrically insulating coating in one embodiment extends along the length of the lead, coating the electrically conductive core. The process provides points of electrical contact at two points along the lead such that in use the lead provides an electrical connection between the points of electrical contact.

The via hole of step b. is provided in the electrically insulating coating order to expose a section of the electrically conductive core. The via in one embodiment does not extend to a significant extent into the electrically conductive core, in one embodiment not more than about 10 μm, in one embodiment not more than about 1 μm, in one embodiment still not more than about 0.1 μm, or in one embodiment the via hole does not extend into the electrically conductive core. The section of the electrically conductive core is in one embodiment exposed in order to allow electrical connection thereto. The area of the electrically conductive core exposed is in one embodiment sufficient to allow electrical contact.

The via hole may be provided mechanically or otherwise. Exemplary methods of providing the via hole are cutting or laser ablation, or in one embodiment laser ablation.

The first electrically conductive material of step c. is introduced into the via hole in order to make contact with the electrically conductive core. In one embodiment, at least a part of the first electrically conductive material occupies at least a part of the volume of the via hole. The first electrically conductive material in one embodiment spans the depth of the via hole in order to allow contact with the further electrically conductive material.

The first electrically conductive material is introduced by sprinkling, dipping, dusting, the use of tweezers, or a combination of two or more selected therefrom, wherein dipping is preferred in one embodiment. In the case of dipping, the coated lead is in one embodiment dipped into electrically conductive particles satisfying on or more of the following:
a. D₅₀ in the range from about 10 to about 100 μm, in one embodiment in the range from about 15 to about 60 μm, or in one embodiment in the range from about 20 to about 50 μm;
b. D₁₀ in the range from about 5 to about 40 μm, in one embodiment in the range from about 10 to about 35 μm, or in one embodiment in the range from about 15 to about 30 μm;
c. D₉₀ in the range from about 40 to about 70 μm, in one embodiment in the range from about 45 to about 65 μm, or in one embodiment in the range from about 50 to about 60 μm;
d. Difference D₉₀-D₁₀ in the range from about 1 to about 25 μm, in one embodiment in the range from about 3 to about 15 μm, or in one embodiment in the range from about 5 to about 10 μm.
e. A standard deviation of diameter in the range from about 0.5 to about 10 μm, in one embodiment in the range from about 1 to about 8 μm, or in one embodiment in the range from about 2 to about 5 μm.

In other embodiment, the following combinations are satisfied: a, b, c, d, a+b, a+c, a+d, b+c, b+d, c+d, a+b+c, a+b+d, a+c+d, b+c+d, a+b+c+d, e, a+e, b+e, c+e, d+e, a+b+e, a+c+e, a+d+e, b+c+e, b+d+e, c+d+e, a+b+c+e, a+b+d+e, a+c+d+e, b+c+d+e or a+b+c+d+e. The features of the first electrically conductive material are for the electrically conductive particles in one embodiment.

The thickness of the first electrically conductive material is in one embodiment the radial distance from the surface of the electrically conductive core to the surface of the first electrically conductive material radially most distant from the electrically conductive core. In one embodiment, the first electrically conductive material includes a particle and the thickness is the diameter of the particle. It is preferred in one embodiment for the first electrically conductive material to be present in the via hole with a thickness which is not significantly less than the depth of the via hole. It is preferred in one embodiment for the thickness of the first electrically conductive material to be sufficient for contact to be made with the further electrically conductive material applied thereto, which is in one embodiment applied mechanically thereto, either with no deformation or with slight deformation of the insulating coating. In one aspect of this embodiment, the thickness of the first electrically conductive material is 0.8 times or more, in one embodiment 0.9 time or more, or in one embodiment 1 time or more the thickness of the insulating coating. In another aspect of this embodiment, the thickness of the first electrically conductive material is greater than the thickness of the insulating coating, in one embodiment at least more than 1, in one embodiment at least about 1.3 times, or in one embodiment at least about 1.5 times the thickness of the insulating coating.

The further electrically conductive material of the step d. is provided in contact with the first electrically conductive material in order to provide an electrical contact via the first electrically conductive material to the electrically conductive core. The further electrically conductive material in one embodiment provides mechanical stability to the contact with the electrically conducting core.

It is preferred in one embodiment for the further electrically conductive material to be applied by solid deformation, in one embodiment including one or more selected from the list consisting of: crimping, notching, bending, twisting, screwing and press fitting, or crimping.

Coated Lead

The coated lead in one embodiment has a core/coating structure. The coated lead in one embodiment has a longitudinal core extending along the lead and a coating which surrounds the core.

In one embodiment the electrically conductive core has a circular cross section. A circular cross section is in one embodiment one which increases the ratio of cross sectional area to cross sectional circumference. In one embodiment the cross sectional area (CSA) divided by the square of the cross sectional circumference (CSC), expressed in the form (CSA)/(CSC)², is at least about 0.06, in one embodiment at least about 0.07, in one embodiment at least about 0.075, or in one embodiment at least about 0.077.

In one embodiment, the insulating coating has a thickness which is less than the radius of the electrically conductive core, in one embodiment having a thickness which is in the range from about 0.01 to about 1 times, in one embodiment in the range from about 0.1 to about 0.95 times, or in one embodiment in the range from about 0.3 to about 0.9 times the radius of the electrically conductive core.

In one embodiment, the electrically conductive core in one embodiment constitutes the major part of the coated lead, in one embodiment by mass or by thickness or both.

In one embodiment, the cross sectional diameter of the lead is in the range from about 0.05 to about 2 mm, in one embodiment in the range from about 0.1 to about 1 mm, in one embodiment in the range from about 0.2 to about 0.8 mm, or in one embodiment in the range from about 0.3 to about 0.7 mm.

Electrically Conductive Core

The electrically conductive core is responsible for electrical conductivity along the lead. The electrically conductive core is in one embodiment of an electrically conductive material, in one embodiment a metallic conductor. The electrically conductive core is in one embodiment a metal. Metals in this context are one or more selected form the group consisting of: an elemental metal and an alloy. Metallic elements in this context are one or more selected from the group consisting of: Pt, Ir, Ta, Pd, Ti, Fe, Au, Mb, Nb, W, Ni and Ti. Alloys in this context include one or more selected from the group consisting of: Pt, Ir, Ta, Pd, Ti, Fe, Au, Mb, Nb, W, Ni and Ti. Alloys in this context are one or more selected from the group consisting of MP35, steel, and platinum/iridium alloy. The steel in one embodiment is stainless steel, in one embodiment a stainless steel suitable for biological applications, in one embodiment 316L, 301 or 304, and in one embodiment 316L. Platinum/iridium alloys in one embodiment have a weight ratio of Pt:Ir in the range from about 60:40 to about 98:2, in one embodiment in the range from about 70:30 to about 95:5, and in one embodiment in the range from about 80:20 to about 90:10.

In one embodiment, the electrically conductive core has a diameter in the range from about 0.01 to about 3 mm, in one embodiment in the range from about 0.013 to about 1.5 mm, in one embodiment in the range from about 0.02 to about 1 mm, or in one embodiment in the range from about 0.05 to about 0.7 mm.

In one embodiment the electrically conductive core is formed of two or more electro-conductive strands, in one embodiment 4 to 9 electrically conductive strands, wherein the strands are in electrical contact.

Electrically Insulating Coating

The purpose of the electrically insulating coating is to provide an electrical insulation between the electrically conductive core and the surroundings. The insulating coating is in one embodiment of an electrically insulating material, in one embodiment a polymer. Polymers in one embodiment are one or more selected from the group consisting of: a poly epoxide, a poly alkene, a polyester and a poly silicon. Polymers in one embodiment are one or more selected from the group consisting of: epoxy resin, silicone, polyurethane, polystyrene, polyethylene, polyethylene terephthalate, polytetrafluoroethylene and ethylene tetra fluoro ethylene. Other polymers which provide an insulating layer may be employed as the insulating coating.

In one embodiment, the electrically insulating coating includes two or more layers of insulating coating, in one embodiment two or more layers of polymer. In one aspect of this embodiment, the insulating coating includes two layers of different electrically insulating material.

The thickness of the electrically insulating coating is in one embodiment in the range from about 3 to about 150 μm, in one embodiment in the range from about 5 to about 100 μm, in one embodiment in the range from about 9 to about 70 μm, and in one embodiment in the range from about 12 to about 40 μm.

First Electrically Conductive Material

The first electrically conductive material provided electrical contact between the electrically conductive core of the lead and the further electrically conductive material. The first electrically conductive material in one embodiment exhibits metallic conductivity. The first electrically conductive material is in one embodiment a metal. Metals in this context are one or more selected form the group consisting of: an elemental metal and an alloy. Metallic elements in this context are one or more selected from the group consisting of: Pt, Ir, Ta, Pd, Ti, Fe, Au, Mb, Nb, W, Ni and Ti. Alloys in this context include one or more selected from the group consisting of: Pt, Ir, Ta, Pd, Ti, Fe, Au, Mb, Nb, W, Ni and Ti. Alloys in this context are one or more selected from the group consisting of MP35, steel, and platinum/iridium alloy. In one embodiment steel is stainless steel, in one embodiment a stainless steel suitable for biological applications, in one embodiment 316L, 301 or 304, and in one embodiment 316L. Platinum/iridium alloys in one embodiment have a weight ratio of Pt:Ir in the range from about 60:40 to about 98:2, in one embodiment in the range from about 70:30 to about 95:5, or in one embodiment in the range from about 80:20 to about 90:10.

In one embodiment, the first electrically conductive material is the same material as the electrically conductive core.

In one embodiment, the first electrically conductive material is introduced into the via hole in the form of one or more particles, in one embodiment a single particle. A particle in one embodiment has a ratio of its largest Cartesian dimension to its lowest Cartesian dimension in the range from about 1 to about 3, in one embodiment in the range from about 1 to about 2.5, more in one embodiment in the range from about 1 to about 2, or in one embodiment in the range from about 1 to about 1.5. The preferred shape of the particles in one embodiment is spherical or significantly spherical. Other shapes of the particles are also possible. In one aspect of this embodiment the particle(s) has/have a shape which increases the ratio of the volume of the particle to the surface area of the particle. It is preferred in one embodiment for the square of the volume of the particle (VOL) divided by the cube of the surface are of the particle (SA), expressed in the form (VOL)²/(SA)³ to be at least about 0.007, in one embodiment at least about 0.008, in one embodiment at least about 0.0083, and in one embodiment at least about 0.0085.

In one embodiment, particles have a mass lower than about 50 μg, in one embodiment lower than about 30 μg, in one embodiment less than about 20 μg, and in one embodiment less than about 10 μg. Particles might have a mass as low as about 0.1 μg or more.

Second Electrically Conductive Material

The second electrically conductive material makes electrical contact with the first electrically conductive material and in this way is in electrical contact with the electrically conductive core. The second electrically conductive material in one embodiment exhibits metallic conductivity. The second electrically conductive material is in one embodiment a metal. Metals in this context are one or more selected form the group consisting of: an elemental metal and an alloy. Metallic elements in this context are one or more selected from the group consisting of: Pt, Ir, Ta, Pd, Ti, Fe, Au, Mb, Nb, W, Ni and Ti. Alloys in this context include one or more selected from the group consisting of: Pt, Ir, Ta, Pd, Ti, Fe, Au, Mb, Nb, W, Ni and Ti. Alloys in this context are one or more selected from the group consisting of MP35, steel, and platinum/iridium alloy. In one embodiment, steel is stainless steel, in one embodiment a stainless steel suitable for biological applications, in one embodiment 316L, 301 or 304, and in one embodiment 316L. Platinum/iridium alloys in one embodiment have a weight ratio of Pt:Ir in the range from about 60:40 to about 98:2, in one embodiment in the range from about 70:30 to about 95:5, or in one embodiment in the range from about 80:20 to about 90:10.

In one embodiment, the second electrically conductive material is of the same material as the electrically conductive core or as the first electrically conductive material or both.

In one embodiment, the second electrically conductive material is a ring, in one embodiment a crimpable ring.

In one embodiment, the second electrically conductive material has a mass in the range from about 0.05 mg to about 0.5 g, in one embodiment in the range from about 0.1 mg to about 0.3 g, or in one embodiment in the range from about 0.2 mg to about 1 mg.

Multiple Electrically Conductive Cores

In one embodiment, the lead includes two or more electrically conductive cores, in one embodiment with at least one of the electrically conductive cores, or in one embodiment all of the electrically conductive cores being electrically insulated from the other electrically conductive core(s). The presence of more than one electrically conductive core in the lead in one embodiment allows the lead to be used to create two or more isolated electrical circuits. In one embodiment, the lead includes two or more electrically conductive cores, electrically insulated from each other, and two or more of the electrically conductive cores, in one embodiment all of the electrically conductive cores, are electrically contacted by a process according to the embodiment. In one aspect of this embodiment, one or more of the electrically conductive cores, in one embodiment all of the electrically conductive cores, is/are connected in two or more placed by a process.

Where the lead includes two or more electrically conductive cores, it is preferred in one embodiment for one or more, in one embodiment all of the electrical contacts made by the process to make contact with a single electrically conductive core.

Where more than one electrically conductive core is present, the features of the electrically conductive core introduced above, apply to at least one of the electrically conductive cores, or in one embodiment all of the electrically conductive cores.

In one embodiment, the coated lead includes two or more coated wires with a core/coating structure, in one embodiment twisted together to form a twisted lead. In one aspect of this embodiment, the ensemble of coated wires if further coated with an electrically insulating material.

Electrically Conductive Particles

A contribution to achieving one or more of the above mentioned objects is made by a use of electrically conductive particles for contacting a lead. According to one embodiment the electrically conductive particles should satisfy one or more of the following criteria:
a. D₅₀ in the range from about 10 to about 100 μm, in one embodiment in the range from about 15 to about 60 μm, and in one embodiment in the range from about 20 to about 50 μm;
b. D₁₀ in the range from about 5 to about 40 μm, in one embodiment in the range from about 10 to about 35 μm, and in one embodiment in the range from about 15 to about 30 μm;
c. D₉₀ in the range from about 40 to about 70 μm, in one embodiment in the range from about 45 to about 65 μm, and in one embodiment in the range from about 50 to about 60 μm;

d. Difference D₉₀-D₁₀ in the range from about 1 to about 25 μm, in one embodiment in the range from about 3 to about 15 μm, and in one embodiment in the range from about 5 to about 10 μm;
e. A standard deviation of diameter in the range from about 0.5 to about 10 μm, in one embodiment in the range from about 1 to about 8 μm, and in one embodiment in the range from about 2 to about 5 μm.

The electrically conductive particles are in one embodiment used as the first electrically conductive material in a process according to one embodiment. In one embodiment, a coated lead is dipped into a portion of the electro-conductive particles, for introducing one or more particles into a via hole in the coating of the coated lead.

The electrically conductive particles are in one embodiment obtained or obtainable by a spray process in which solid particles, in one embodiment spherical or essentially spherical particles, are obtained through the solidification of molten material in a spray.

It may be necessary to obtain a portion of electrically conductive particles with a modified particle size distribution. One embodiment method is sieving.

Medical Devices

A contribution to achieving one or more of the above mentioned objects is made by a medical device including a contacted lead according to one embodiment. Medical devices in this context are one or more selected form the group consisting of: a pacemaker, a defibrillator, a cardo resynchronisation device, a neuro-stimulator and a measuring device.

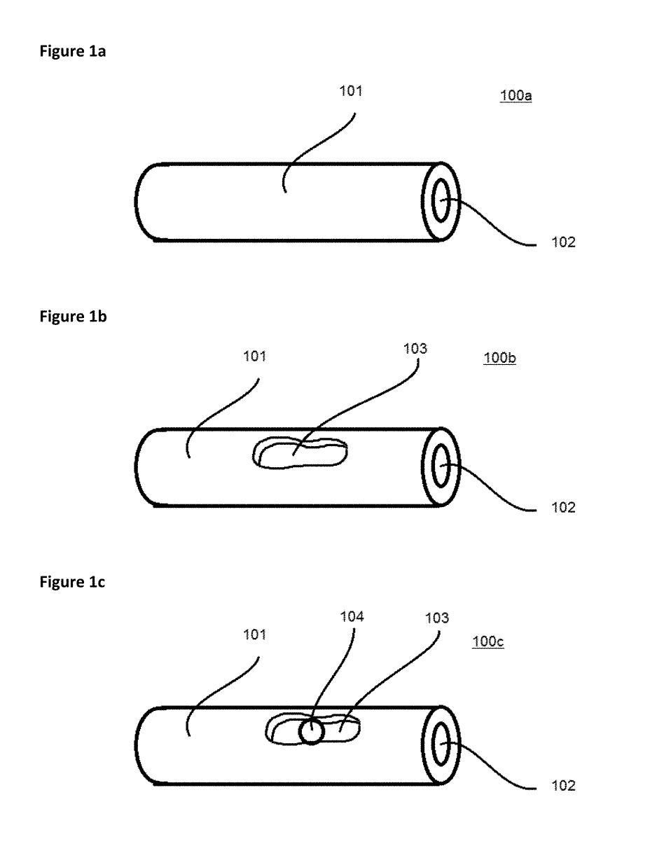

FIG. 1a illustrates a coated lead 100a according to one embodiment. The coated lead 100a includes an electrically conductive core 102 of electrically conductive material coated with an electrically insulating coating 101 of electrically insulating material. In this case, the electrically conductive material is a rhodium/platinum alloy (20% rhodium and 80% platinum, by mass) and the electrically conductive core has a diameter of 0.5 mm. In this case, the electrically insulating coating is 20 μm thick and the electrically insulating coating is of polyurethane.

FIG. 1b illustrates 100b a coated lead with via hole 103. The coated lead with via hole 100b was obtained from the coated lead 100a by laser ablation. The via hole 103 extends through the electrically insulating coating 101 to expose a section of the surface of the electrically conductive core with a cross sectional area of 0.02 mm².

FIG. 1c illustrates 100c a coated lead with a via hole 103 and a particle 104 in the via hole 103. The particle 104 is of an electrically conductive material, is spherical and has a diameter of 40 μm. The particle 104 is in electrical contact with the electrically conductive core 102. The particle 104 has been introduced into the via hole 103 using tweezers. In this case, the particle is of rhodium/platinum alloy (20% rhodium and 30% platinum, by mass).

FIG. 1 c′ illustrates 100c in which a total of three particles 104a, 104b & 104c have been introduced into the via hole.

FIG. 1d illustrates 100d a coated lead with a via hole 103, a particle in the via hole and an electrically conductive ring 105 located over the via hole and particle. The conductive ring has been crimped around the lead so as to cover the via hole and the particle and to make electrical contact with the particle. In this case, the conductive ring is of rhodium/platinum alloy (20% rhodium and 80% platinum, by mass), and has a thickness of 0.5 mm.

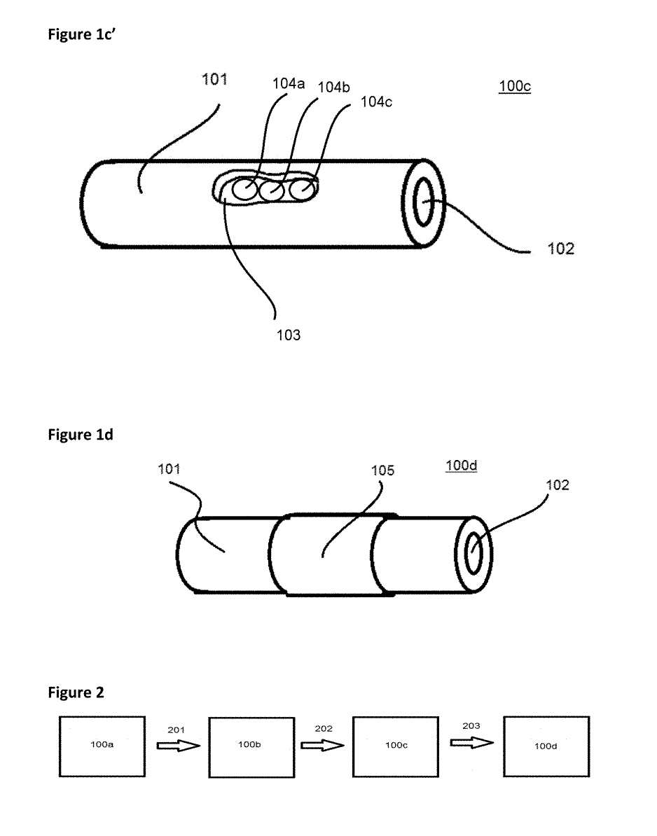

FIG. 2 illustrates schematically a process according to one embodiment for contacting a coated lead. FIG. 2 illustrates how the items 100a, 100b, 100c & 100d are related by process steps. In step a), a coated lead 100a is provided. In step b) 201, a via hole is provided in the insulating coating of the coated lead 100a to obtain a coated lead with a via hole 100b. In step c) 202, a particle is introduced into the via hole to obtain a coated lead with a via hole and a particle in the via hole 100c. In step d) 203, an electrically conductive ring is crimped over the via hole and the particle to obtain the electrically contacted lead 100d.

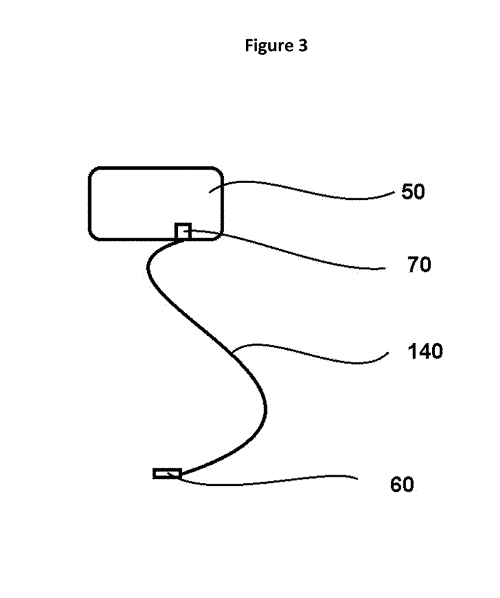

FIG. 3 illustrates schematically a pacemaker 50 with a pulse generator 70, and a lead 140 including an electrode 60. The lead 140 connects the pulse generator 70 and the heart tissue via the electrode 60. The lead 140 has been electrically connected to the pulse generator 70 and the electrode 60 by a process according to one embodiment.

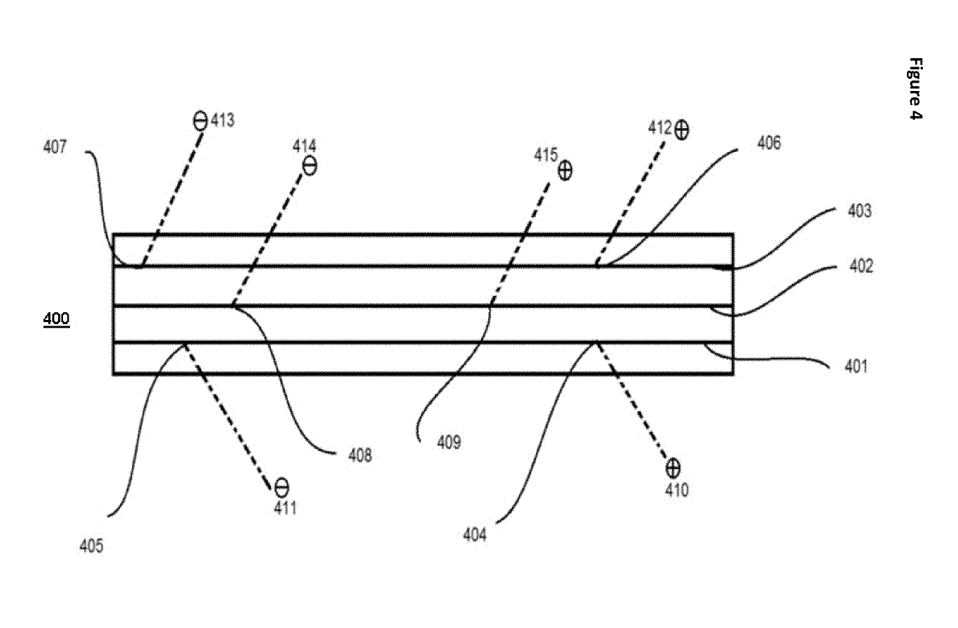

FIG. 4 illustrates schematically a lead 400, including 3 electrically conductive cores 401, 402 & 403. Electrically conductive core 401 is electrically connected according to in one embodiment at points 404 and 405 to allow an electrical circuit between notional terminals 410 and 411 respectively. Similarly, electrically conductive core 402 is electrically connected according to one embodiment at points 408 and 409 to allow an electrical circuit between notional terminals 414 and 415 respectively. Similarly, electrically conductive core 403 is electrically connected according to one embodiment at points 406 and 407 to allow an electrical circuit between notional terminals 412 and 413 respectively.

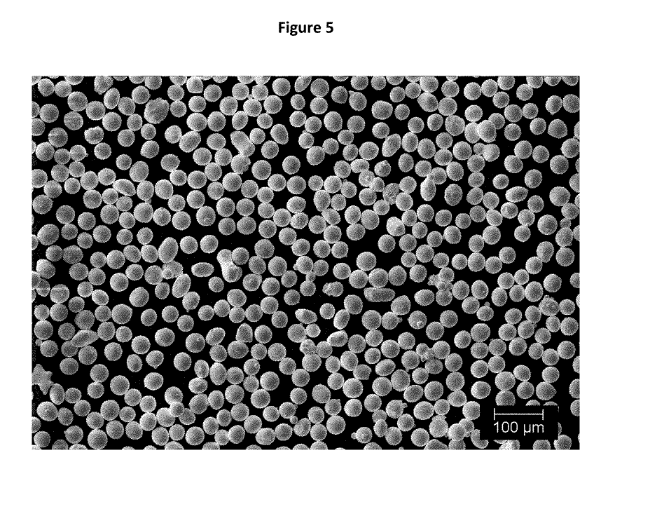

FIG. 5 illustrates a scanning electron microscope image of a portion of electrically conductive particles according to one embodiment. The particles are of rhodium/platinum alloy (20% rhodium, 80% platinum, by mass) and have D₅₀=38 μm, D₁₀=35 μm, D₉₀=41 μm.

Test Methods

Surface Area of Particles

BET measurements to determine the specific surface area of electrically conductive particles are made in accordance with the static volumetric method according to DIN ISO 9277:2010. A Gemini 2390 (from Micromeritics) which works according to the SMART method (Sorption Method with Adaptive dosing Rate), is used for the measurement. As reference material Alpha Aluminum oxide CRM BAM-PM-102 available from BAM (Bundesanstalt für Materialforschung und-prüfung) is used. Filler rods are added to the reference and sample cuvettes in order to reduce the dead volume. The cuvettes are mounted on the BET apparatus. The saturation vapour pressure of nitrogen gas (N₂ 5.0) is determined. A 1 g sample is weighed into a glass cuvette. The sample is kept at 100° C. for 2 hours in order to dry it. After cooling the weight of the sample is recorded. The glass cuvette containing the sample is mounted on the measuring apparatus. To degas the sample, it is evacuated at a pumping speed selected so that no material is sucked into the pump. The mass of the sample after degassing is used for the calculation. The dead volume is determined using Helium gas (He 4.6). The glass cuvettes are cooled to 77 K using a liquid nitrogen bath. For the adsorptive, N₂ 5.0 with a molecular cross-sectional area of 0.162 nm² at 77 K is used for the calculation. A multi-point analysis with 5 measuring points is performed and the resulting specific surface area given in m²/g.

Fatigue Test

Fatigue resistance is measured according to the test described in “prEN 45502 Parts 2 & 3 CEN/CENELEC, Active Implantable Medical Devices—Brady and Tachy Lead Tests Draft/Standard”.

### Examples

### Example 1 (Inventive)—Contacting of a Lead According to One Embodiment

Electrical contacts were made to 15 cm long leads having a 0.2 mm diameter core of rhodium/platinum alloy (20% rhodium, 80% platinum, by mass) and a 20 μm coating of PTFE. 1 cm from each end of the lead, an electrical contact was made as follows: First, a via hole was made through the PTFE coating by laser ablation using a Varydisk laser available from Dausinger+Giesen GmbH. The via hole had a circular cross section of 60 μm diameter. Second, a portion of spherical rhodium/platinum alloy (20% rhodium, 80% platinum, by mass) particles with D₅₀=38 D₁₀=35 D₉₀=41 μm, commercially available from Heraeus Deutschland GmbH & Co. KG, was provided and the lead was dipped into the portion of particles in order to introduce a particle into the via hole. Third, a cylindrical ring of rhodium/platinum alloy (20% rhodium, 80% platinum, by mass) with outer diameter of 350 μm, an inner diameter of 260 μm and a cylinder length of 500 μm, available from Heraeus Deutschland GmbH & Co. KG, was threaded over the lead, positioned so as to cover the via hole with introduced particle and crimped tight onto the lead using a multi-jaw chuck.

### Example 2a (Comparative)—Contacting of a Lead by Soldering

The lead was provided as in example 1 and electrical contact was made 1 cm from each end of each lead as follows: First, a via hole was made through the PTFE coating by laser ablation using a Varydisk laser available from Dausinger+Giesen GmbH. The via hole had a circular cross section of 60 μm diameter. Second, a cylindrical ring as in example 1 was threaded over the lead, positioned so as to cover the via hole and a contact was made between the ring and the core of the lead by soldering with gold at 700° C.

### Example 2b (Comparative)—Contacting of a Lead by Soldering

The lead was provided as in example 1 and electrical contact was made 1 cm from each end of each lead as follows: First, a via hole was made through the PTFE coating by laser ablation using a Varydisk laser available from Dausinger+Giesen GmbH. The via hole had a circular cross section of 60 μm diameter. Second, a cylindrical ring as in example 1, but with a 60 μm via hole was provided. The ring was threaded over the lead and positioned such that the via in the ring was over the via in the coating of the lead. Contact was made between the ring and the core of the lead by soldering through the two superimposed via holes with gold at 700° C.

### Example 3 (Comparative)—Contacting of a Lead by Crimping

A lead was provided as in example 1 and electrical contact was made 1 cm from each end of each lead as follows: First, a cylindrical ring as in example 1 was threaded over the lead and positioned 1 cm from the end of the lead. Second, the ring was crimped using a multi-jaw chuck to contact the ring with the core of the lead.

Properties of the Contacted Leads

For each of the examples (1, 2a, 2b & 3), 5 leads were contacted as described. Each contacted lead was tested for fatigue resistance. The success rate of contacting the leads and the results for fatigue resistance are illustrated in table 1.

TABLE 2

| Example | Successful contacting? | Fatigue resistance |
|---|---|---|
| 1 | Yes, in all 5 samples | ++ |
| 2a | 2 leads contacted, 3 failed | −− |
| 2b | 3 leads contacted, 2 failed | −− |
| 3 | 4 leads contacted, 1 failed | −− |

++ Very good fatigue resistance (no failures after 400,000,000 cycles),
−− very poor fatigue resistance (failed after an average of less than 300,000 cycles)

What is claimed is:

1. A method for electrically contacting a coated lead comprising:
a. providing a coated lead comprising an electrically conductive core and an electrically insulating coating;
b. providing a via hole in the electrically insulating coating in order to expose a section of the electrically conductive core;
c. introducing a first electrically conductive material into the via hole such that it contacts at least a part of the exposed section of the electrically conductive core; and
d. applying a second electrically conductive material to the coated lead such that it contacts at least a part of the first conductive material.

2. The method of claim 1, wherein the first electrically conductive material comprises an electrically conductive particle.

3. The method of claim 2, wherein the particle has a diameter in the range from about 5 to about 500 μm.

4. The method of claim 2, wherein the introduction of the particle comprises sprinkling, dipping, dusting, the use of tweezers, or a combination of two or more selected therefrom.

5. The method of claim 1, wherein the first electrically conductive material comprises a metal.

6. The method of claim 1, wherein the second electrically conductive material is applied by solid deformation.

7. The method of claim 1, wherein the lead has a diameter less than about 2 mm.

8. The method of claim 1, wherein the lead comprises 2 or more electrically conductive cores.

9. The method of claim 8, wherein 2 or more of the electrically conductive cores are electrically insulated from each other by an insulated material.

10. The method of claim 1, wherein the via hole exposes a single conductive core.

11. The method of claim 1, wherein the provision of the via hole in step b. is performed with a laser.

12. The method of claim 1, wherein the lead is bio-compatible and configured for use as a thin lead in a medical device.

13. An electrically contacted lead obtainable by a method of claim 1.

14. An electrically contacted lead comprising:
a. an electrically conductive core
b. an electrically insulating coating which coats the electrically conductive core;
c. a via hole in the electrically insulating coating which exposes a section of the electrically conductive core;
d. a first electrically conductive material in the via hole which contacts at least a part of the exposed section of the electrically conductive core; and
e. a further electrically conductive material which contacts at least a part of the first conductive material.

15. A medical device comprising a contacted lead according to claim 14.

16. A use of electrically conductive particles for contacting a coated lead, wherein the electrically conductive particles satisfy one or more of the following criteria:
a. D₅₀ in the range from about 10 to about 100 μm;
b. D₁₀ in the range from about 5 to about 40 μm;
c. D₉₀ in the range from about 40 to about 70 μm;
d. Difference D₉₀-D₁₀ in the range from about 1 to about 25 μm; and
a standard deviation of diameter in the range from about 0.5 to about 10 μm.
